(19) United States
(12) Patent Application Publication
(10) Pub. No.: US 2005/0125853 A1
(43) Pub. Date: Jun. 9, 2005

(54) Method for generating genetically modified animals

(75) Inventor: Vishwas Pravinchandra Parekh, Memphis, TN (US)

Correspondence Address: ST. JUDE CHILDREN'S RESEARCH HOSPITAL;OFFICE OF TECHNOLOGY LICENSING, 332 N. LAUDERDALE, MEMPHIS, TN, 38105, US

(73) Assignee: St. Jude Children's Resarch Hospital, Memphis, TN (US)

(21) Appl. No.: 10/505,760

(22) PCT Filed: Mar. 10, 2003
(86) PCT No.: PCT/US03/07304

Related U.S. Application Data
(60) Provisional application No. 60/366,685, filed on Mar. 22, 2002.

(51) Int. Cl. A01K 67/027; C12N 15/85

(52) U.S. Cl. 800/21; 435/455

(57) ABSTRACT

The present invention represents an improvement upon the basic method of generating genetically modified animals, particularly knock-outs, through the injection or aggregation of genetically modified foreign cells with wild type blastocysts or embryos. This improvement comprises the use of animals with permanent or conditional fertility defects as the source of blastocysts/embryos in this process. By using such animals, germ cells derived from the genetically modified foreign cells have a competitive advantage in the process and the frequency of producing genetically modified animals is increased. Chimeric blastocysts or aggregates produced according to this improved method are also part of the invention. In another aspect, the invention teaches the generation and use of an inducibly sterile animal. A method for rendering wild type blastocysts or early stage embryos sterile for reproduction is also contemplated as part of the invention.

### FIELD OF THE INVENTION

This invention relates to methods for producing genetically modified animals, particularly knock-out animals in which a particular gene of interest has been rendered nonfunctional. The invention also relates to methods for the incorporation of a transgene or other genetic modification into the germ line of an animal so that it can be inherited.

### BACKGROUND

Genetically modified animals are used for a variety of purposes in modern biologic research. They include (a) transgenic animals, which either possess a foreign gene from other biologic species or a native gene from same species but in a modified form in terms of its structure, genomic location, expression pattern or regulatory mechanisms, (b) knock-out animals, in which one or more endogenous genes have been rendered fully, partially or conditionally nonfunctional, (c) knock-in animals, in which a transgene is placed under the control of regulatory sequences of an endogenous gene, and (d) a broad category of animals showing chromosomal rearrangements, deletions, point mutations and many other varieties of natural or artificial genomic alterations.

Among these, knocking out a gene and observing the resultant phenotype is the best and most authentic available method for analyzing gene function. In addition, producing conditional mutants, point mutations and knock-ins are indispensable for finding out the mechanisms of gene function at various stages of development, in different cellular and organ environments and in analyzing complex protein-nucleic acid and protein-protein interactions, eventually leading to deeper understanding of biologic systems, genetic and otherwise disease mechanisms and discovery of drug targets.

A standard approach for creating a knock-out mouse (and sometimes transgenic mice and other mutants) involves the injection of blastocysts from wild type mice with genetically modified embryonic stem (ES) cells. Mice derived from the injected blastocysts will typically be chimeric with some cells derived from the original donor blastocyst and others derived from the injected ES cells. An unpredictable number of these chimeric mice will have a germ line contribution from the injected ES cells. These chimeras are further bred to generate fully ES cell derived mice, heterozygous or homozygous for the genetic modification in the injected ES cells. “Gene Knockout Protocols, Methods in Molecular Biology”, vol 158, edited by Martin J. Tymms and Ismail Kola, Human Press, New Jersey (2001); “Gene targeting: A practical approach”, 2^{nd} edition, edited by A. L. Joyner, Oxford University Press, Inc, New York (2000).

A modification in this standard approach is to aggregate wild type donor morula or eight cell stage embryos with the genetically modified ES cells and allowing them to develop to the chimeric blastocyst stage in-vitro. “Gene Knockout Protocols, Methods in Molecular Biology”, vol 158, edited by Martin J. Tymms and Ismail Kola, Human Press, New Jersey (2001); “Gene targeting: A practical approach”, 2^{nd} edition, edited by A. L. Joyner, Oxford University Press, Inc, New York (2000). Since the advent of this technology over a decade ago [Capecchi M R., “Altering the genome by homologous recombination” Science 244(4910): 1288-92 (1989) Mansour S L et al., “Disruption of the proto-oncogene int-2 in mouse embryo-derived stem cells: a general strategy for targeting mutations to non-selectable genes” Nature 336(6197): 348-52 (1988)], about 2000 unique gene knock-outs have been described. Mouse Knock-out and Mutation Database located on the internet at http://research.bmn.com/mkmd.

A significant handicap of these standard approaches is that incorporation of genetically modified ES cells into the germ cell lineage (i.e germline transmission) is infrequent and unpredictable. Success of these approaches depends upon (1) the number of injected/aggregated ES cells which actually differentiate in the growing blastocyst along the germ cell lineage, (2) the resulting ratio of ES cell derived and blastocyst derived primordial germ cells, and (3) competition among ES cell derived and blastocyst derived gametes for fertilization during the breeding step. Large numbers of blastocysts are injected or large numbers of embryos are aggregated with ES cells and subsequently large numbers of resulting animals are screened and bred in order to overcome this problem and find the occasion where the genetically modified ES cells have successfully been incorporated into the germ cell lineage and have generated functional germ line cells that have effectively competed with wild type blastocyst derived germ line cells in the reproduction process.

### SUMMARY OF THE INVENTION

In one aspect, the present invention represents an improvement upon the basic methods of generating genetically modified animals from genetically modified foreign cells such as embryonic stem (ES) cells, embryonic germ (EG) cells, teratocarcinoma stem cells and primordial germ cells that are capable of developing into germ line cells. The genetically modified animals are generated in these methods through the injection of wild type blastocysts with genetically modified foreign cells or through the aggregation of wild type embryos with genetically modified foreign cells. The improvement taught by the invention comprises the use of animals with either permanent or inducible fertility or other germ line defects as the source of blastocysts or embryos in this process. By using such animals as the source of blastocysts or embryos in chimeras generated by injection or by aggregation, germ line cells derived from the genetically modified foreign cells have a competitive advantage in the development and reproduction process and the frequency of producing genetically modified animals is increased.

The present invention also provides a method for generating a chimeric blastocyst or chimeric aggregate by injecting a blastocyst or aggregating early stage embryos from a source animal that has a defect in its germ line cells with foreign cells having a desired genetic modification. The resulting chimeric blasotcyst or aggregate is also considered a part of the present invention. Such a chimeric blastocyst or aggregate is useful in faster and more efficient generation of a whole animal having the desired genetic modification.

In another aspect, the present invention provides a method for generating a chimeric blastocyst or chimeric aggregate by injecting or aggregating wild type diploid or tetraploid embryos with foreign cells having a desired genetic modification and foreign cells that have a defect in fertility related gene/s. The resulting chimeric blasotcyst or aggregate formed by the two foreign cell types is also considered a part of the present invention. Such a chimeric blastocyst or aggregate can grow into a chimeric animal with viable germ line cells derived from foreign cells having the desired genetic modification, which in turn can be used to generate whole animals homozygous or heterozygous for the desired genetic modification.

In yet another aspect, the invention provides a method for creating an inducibly sterile mouse. This method utilizes a conventional approach for creating inducibly mutated mice for certain genes. Kistner, A. et al., “Doxycycline-mediated quantitative and tissue-specific control of gene expression in transgenic mice”, Proc Natl Acad Sci U S A. 93(20):10933-8 (1996). In accordance with this method, a mouse is genetically engineered such that one or more genes required for fertility can be regulated or conditionally mutated using inducible promoter system(s). Such a mouse can be bred to generate all (100%) homozygous blastocysts/early stage embryos mutated for the fertility related gene(s) in contrast to 75% or less homozygous mutant blastocysts/embryos generated from mice with fertility related genetic defects described thus far. This inducibly mutated mouse may be used as an excellent source of blastocysts or embryos in the methods of the invention.

In another aspect, the invention provides a method for rendering wild type blastocysts or early stage embryos sterile for reproduction by incubating or injecting them with certain substances including but not limited to certain chemicals, antibodies and anti-sense RNA molecules which render the fertility related gene/s or gene products dysfunctional.

### DETAILED DESCRIPTION OF THE INVENTION

Definitions:

The following terms and phrases used in this application are intended to have the following meanings:

“Blastocyst” means an early developmental stage of embryo comprising of inner cell mass (from which embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000)

“Conditional Knock-out” or “Conditional mutation” means when the knock-out or mutation is achieved when certain conditions are met. These conditions include but are not limited to presence of certain inducing agents, recombinases, antibiotics, and certain temperature or salt levels.

“Early stage embryo” means any embryo at embryonic stages between fertilized ovum and blastocyst. Typically, eight cell stage and morula stage embryos are referred to as early stage embryos.

“Embryonic germ cells” or “EG cells” means primordial germ cell derived cells which have the potential to differentiate into all the cell types of body and are as amenable to genetic modification as Embryonic stem cells, to the extent that sometimes the distinction between EG cells and ES cells is ignored. “Developmental Biology”, sixth edition, ed. by Scott F. Gilbert, Sinauer Associates, Inc., Publishers, Sunderland, Mass. (2000).

“Embryonic stem cells” or “ES cells” means cultured cells derived from inner cell mass of early stage embryo, which are amenable to genetic modification and which retain their totipotency and can contribute to all organs of resulting chimeric animal if injected into host embryo. “Developmental Biology”, sixth edition, ed. by Scott F. Gilbert, Sinauer Associates, Inc., Publishers, Sunderland, Mass. (2000).

“Fertilization” means the union of male and female gametes during reproduction resulting into formation of zygote, the earliest developmental stage of an embryo. “Foreign cell” means any cell that can be genetically modified or can be derived from a genetically modified cell and that can contribute towards the germ line of a chimeric embryo when injected or aggregated with a donor blastocyst/embryo. This includes, but is not limited to, embryonic stem (ES) cells, teratocarcinoma stem cells, primordial germ cells, and embryonic germ (EG) cells.

“Genetically modified” means those animals or embryos or cells which have a desired genetic modification such as a knock-out, knock-in, conditional, inducible, transient or point mutation(s) of any gene or its regulatory mechanism or a transgenic with foreign or modified gene/s or regulatory sequences, or having undergone genomic modification in any way including but not limited to recombination, chromosomal deletion, addition, translocation, rearrangement or addition, deletion or modification of nucleic acid, protein or any other natural or synthetic molecule/s or organelle, or cytoplasmic or nuclear transfer, leading to inheritable changes.

“Germ cell development” means the process by which certain cells in the early stage developing embryo differentiate into primordial germ cells.

“Germ cell migration” means the process by which primordial germ cells, after originating in the extraembryonic mesoderm travel back in the embryo through allantois (precursor of umbilical cord) and continue to migrate through adjacent yolk sac, hindgut, and dorsal mesentery to finally reach the genital ridge (developing gonad). “Developmental Biology”, sixth edition, ed. by Scott F. Gilbert, Sinauer Associates, Inc., Publishers, Sunderland, Mass. (2000).

“Germ line cell” means any cell, at any stage of differentiation towards mature gametes, including mature gametes.

“Knock-in” means replacement of an endogenous gene with a transgene or with same endogenous gene with some structural modification/s, but retaining the transcriptional control of the endogenous gene.

“Knock-out” means disruption of the structure or regulatory mechanism of a gene. Knock-outs may be generated through homologous recombination of targeting vectors, replacement vectors or hit-and-run vectors or random insertion of a gene trap vector resulting into complete, partial or conditional loss of gene function. “Oogenesis” means the process of generation of mature eggs from the primordial germ cells in females.

“Primordial germ cells” means those cells arising early in the embryonic development that finally give rise to mature gametes in both sexes.

“Spermatogenesis” means the process of generation of mature sperms from the primordial germ cells in males.

“Teratocarcinoma stem cells” means those cells derived from teratocarcinomas, which retain their totipotency and are as amenable to genetic modification as ES cells and that can contribute to all cells in body, including germ line cells, if chimeric animals are generated using them. “Developmental Biology”, sixth edition, ed. by Scott F. Gilbert, Sinauer Associates, Inc., Publishers, Sunderland, Mass. (2000).

“Tetraploid embryos” means embryos comprising of cells that have a tetraploid genome rather than usual diploid genome.

“Transgenic animal” means an animal that harbors a foreign gene(s), which is either from other biologic species or is from the same species but in the modified form in terms of its structure, pattern of expression or regulatory mechanisms.

“Wild type” means those animals and blastocysts, embryos or cells derived therefrom, which have not been genetically modified and are usually inbred and outbred strains developed from naturally occurring strains.

### Description

In one aspect, the present invention represents an improvement upon the conventional process of generating a genetically modified animal having a desired genetic modification from wild type blastocysts injected with foreign cells, particularly ES cells. In this process, the foreign cells possess the desired genetic modification. The genetically modified animal is made when at least some of the germ line cells in the animal generated from the blastocyst are derived from the injected foreign cells and these germ line cells transmit the desired genetic modification to offspring. The same objective can also be achieved by aggregating wild type early stage embryos with genetically modified foreign cells. “Gene Knockout Protocols, Methods in Molecular Biology”, vol 158 edited by Martin J. Tymms and Ismail Kola, Human Press, New Jersey (2001); “Gene targeting: A practical approach”, 2^{nd} edition, edited by A. L. Joyner, Oxford University Press, Inc, New York (2000).

The improvement represented by the present invention relates to the source of blastocysts or embryos used in this process. Typically, wild type animals with normal reproductive systems are used as the source for these blastocysts or embryos. However, by using animals, which are compromised in their reproductive ability as the source of blastocysts or embryos, the rate of generation of genetically modified animals from this method can be increased according to the present invention.

A large number of fertile chimeric animals produced from injected blastocysts or aggregated embryos using the conventional approach may not have a sufficient number of germ line cells derived from the injected foreign cells to produce offspring having the desired genetic modification. In order to distinguish these animals from chimeras which do have a sufficient number of germ line cells derived from the injected foreign cells, a large number of chimeric animals must be bred and their offspring selected or screened for the desired genetic modification. This process can take several weeks with no guarantee of success, particularly in those cases where the number of chimeric animals and/or degree of chimerism resulting from an injection/aggregation experiment is low. In such cases, the low number of germ line cells derived from injected ES cells may not be able to effectively compete with large numbers of wild type germ line cells derived from the donor blastocyst/embryo to generate offspring.

In contrast, the majority, if not all, of the fertile chimeric animals produced from injected blastocysts or embryos using the approach taught herein are expected to have functional germ line cells derived entirely or predominantly from the injected foreign cells and produce most, if not all, offspring having the desired genetic modification. This increases the chances of successful germ line transmission of the desired genetic modification and also increases the yield of heterozygous offspring having the desired genetic modification from the first breeding of the chimeric animals, thereby allowing a large colony of foreign cell derived animals having the desired genetic modification to be established more quickly and easily. If a blastocyst/embryo is used from an animal that cannot produce functional/viable spermatozoa, the practitioner can determine if an injection experiment has been successful at an earlier stage simply based on the ability of male chimeras to impregnate female breeding partners. The same is true for animals that cannot produce functional/viable eggs with respect to the ability of female chimeras to become impregnated by male breeding partners. The same is also true for animals that cannot produce functional/viable primordial germ cells and animals whose mature gametes do not fertilize efficiently.

Animals which may be used as a source of blastocysts or embryos according to the invention include any animal having a defect such that no germ line cells develop or very few germ line cells develop or that germ line cells derived therefrom will not be viable or which will suffer a reproductive disadvantage relative to germ line cells derived from a foreign cell. Preferred animals will have a permanent or conditional defect in spermatogenesis, oogenesis, germ cell development, germ cell migration, or fertilization. Particularly preferred are animals having a defect in spermatogenesis. These preferred animals have the following features:
The defect is inherent to germ line cells themselves and not in the testicular, ovarian or embryonic environment; and
The adult homozygous animals do not have any gonadal or hormonal defects, which make them completely incapable of reproduction.

Animals useful in the present invention which have a defect in spermatogenesis include, but are not limited to, animals which have permanent or conditional mutations in the following genes or in their regulatory mechanism(s):
Calmegin as described in Ikawa, M. et al., “Calmegin Is Required for Fertilin Heterodimerization and Sperm Fertility”, Dev Biol. 240(1): 254-61 (December 2001);
Fertilin beta as described in Cho, C. et al., “Fertilization Defects in Sperm from Mice Lacking Fertilin”, Science 281: 1857-1859 (1998); Nishimura, H. et al., “Analysis of Loss of Adhesive Function in Sperm Lacking Cyritestin or Fertilin”, Dev. Biol. 233(1):204-13 (May 2001);
CatSper as described in Ren, D. et al., “A sperm ion channel required for sperm motility and male fertility”, Nature 413(6856):603-9 (October 2001);
Cyclin A1 as described in Liu, D. et al., “Cyclin A1 is required for meiosis in the male mouse” Nat Genet. 20(4):377-80 (1998);
Hsp 70-2 as described in Dix, D. J. et al., “Targeted gene disruption of Hsp70-2 results in failed meiosis, germ cell apoptosis, and male infertility”, Proc. Natl. Acad. Sci. 93: 3264-3268 (1996);
Scp3 as described in Yuan, L. et al., “The Murine SCP3 Gene Is Required for Synaptonemal Complex Assembly, Chromosome Synapsis, and Male Fertility”, Mol Cell 5(1): 73-83 (2000);
A-myb as described in Toscani, A. et al., “Arrest of spermatogenesis and defective breast development in mice lacking A-myb”, Nature 386(6626): 713-717 (1997);
ACE as described in Krege, J. H. et al., “Male-female differences in fertility and blood pressure in ACE-deficient mice”, Nature 375(6527): 146-148 (1995);
Ahch as described in Yu, R. N. et al., “Role of Ahch in gonadal development and gametogenesis” Nat Genet 20(4):353-7 (1998);
Dazl as described in Bianca, H. G. J. et al., “Nature of the Spermatogenic Arrest in Dazl -/- Mice”, Biol Reprod 65: 771-776 (2001);
Trf2 as described in Zhang, D. et al., “Spermiogenesis Deficiency in Mice Lacking the Trf2 Gene”, Science 292(5519):1153-5 (May 2001);
Jsd as described in Ohta, H. et al., “Defect in germ cells, not in supporting cells, is the cause of male infertility in the jsd mutant mouse: proliferation of spermatogonial stem cells without differentiation”, Int. J. Androl. 24(1):15-23 (February 2001); and
Protamine-1 and -2 as described in Cho, C. et al., “Haploinsufficiency of protamine-1 or -2 causes infertility in mice”, Nat. Genet. 28(1):82-6 (May 2001).

Currently available mutant mice strains that have defect/s in reproductive ability may also be used as the source of blastocysts or embryos in the present invention. This includes but is not limited to following strains:
Morc as described in Watson, M. L. et al., “Identification of morc (microrchidia), a mutation that results in arrest of spermatogenesis at an early meiotic stage in the mouse”, Proc. Natl. Acad. Sci. 95: 14361-14366 (1998);
W/Wv as described in Kuroda, H. et al., “Differentiation of germ cells in seminiferous tubules transplanted to testes of germ cell-deficient mice of W/Wv and Sl/Sld genotypes”, J. Cell. Physiol. 139(2): 329-34 (1989);
Sl/Sld as described in Kuroda, H. et al., “Differentiation of germ cells in seminiferous tubules transplanted to testes of germ cell-deficient mice of W/Wv and Sl/Sld genotypes”, J. Cell. Physiol. 139(2): 329-34 (1989); and
XoSxrb as described in Sutcliffe, M. J. and Burgoyne, P. S., “Analysis of the testes of H—Y negative XOSxrb mice suggests that the spermatogenesis gene (Spy) acts during the differentiation of the A spermatogonia” Development 107(2): 373-80 (1989).

Optimally, the animal used as the source of blastocyst/embryo carries a permanent or conditional defect/s at any level/s in spermatogenesis. Such defects include homozygous mutations in genes like Calmegin, Fertilin beta, CatSper, Cyclin A1, Hsp 70-2, Scp3, A-myb, ACE, Ahch, Dazl, Trf2 and Jsd, and heterozygous mutations in protamine-1 and protamine-2, as well as mutants like Morc, XoSxrb, W/Wv and Sl/Sld.

Animals useful in the present invention which have a defect in oogenesis and/or fertilization include, but are not limited to, animals which have permanent or conditional mutations in the Cd-9 gene as described in Kaji, K. et al., “The gamete fusion process is defective in eggs of Cd9-deficient mice”, Nat. Genet. 24(3): 279-82 (2000) or in its regulatory mechanism(s).

Animals useful in the present invention, which have a defect in germ cell development and/or germ cell migration include, but are not limited to, animals which have permanent or conditional mutations in the following genes or in their regulatory mechanism(s):
Smad1 as described in Tremblay, K. D. et al., “Mouse embryos lacking Smad1 signals display defects in extra-embryonic tissues and germ cell formation”, Development 128: 3609-3621 (2001);
Smad5 as described in Chang H. and Matzuk, M. M., “Smad5 is required for mouse primordial germ cell development”, Mech. Dev. 104(1-2): 61-7 (June 2001);
Bmp4/Bmp8b as described in Ying, Y. et al., “Induction of primordial germ cells from murine epiblasts by synergistic action of BMP4 and BMP8B signaling pathways”, Proc. Natl. Acad. Sci. 98: 7858-7862 (2001);
E-cadherin as described in Di Carlo, A. and De Felici, M., “A Role for E-cadherin in mouse primordial germ cell development”, Dev. Biol. 226(2): 209-19 (2000);
Fancc as described in Nadler, J. J. and Braun, R. E., “Fanconi anemia complementation group C is required for proliferation of murine primordial germ cells” Genesis 27(3): 117-23 (2000);
Bcl-x/Bax as described in Rucker, E. B. 3^{rd} et al., “Bcl-x and Bax regulate mouse primordial germ cell survival and apoptosis during embryogenesis”, Mol Endocrinol. 14(7): 1038-52 (2000); and
Beta1-integrin as described in Anderson, R. et al., “Mouse primordial germ cells lacking beta1 integrins enter the germline but fail to migrate normally to the gonads”, Development 126: 1655-1664 (1999).

Animals useful as a source of blastocysts or early stage embryos in the present invention may be obtained from the laboratories that have reported the phenotype arising from respective gene knock-outs. One can also generate such animals by creating an ES cell line with a permanent or conditional mutation in one or more of the genes identified above or in their regulatory mechanisms or by screening for naturally occurring mutants that have the desired characteristics. Alternatively, many such animals are available from existing repositories such as The Jackson Laboratory, Bar Harbor, Me.

In any of the above described gene mutations, if the resulting animal shows phenotypic defect/s in addition to the primary fertility defect/s, they may be less preferable for use in the methods of the invention. Where this occurs, conditional mutants for the respective gene(s) can be produced such that the gene mutation only occurs at a certain developmental stage or only in certain cell types, thereby avoiding the additional abnormal phenotype(s) observed in the corresponding non-conditional mutant. Conventional methods may be used to generate such conditional mutant mice.

When using the fertility defective mice described in this invention as the source animals, it may be desirable to genotype them prior to breeding to ensure that a homozygous mutant fertile partner is bred with a heterozygous mutant partner of the sex that shows homozygous sterile phenotype. This prior selection generates the maximum number of homozygous mutant blastocysts/embryos. Even after this selection process, about 25% of the blastocysts/embryos generated are expected to be heterozygous for the gene mutation, resulting into about 25% chimeras that do not give the desired enrichment of germ line cells derived from foreign cells having a desired genetic modification. To identify and exclude such chimeras from further breeding process, one can associate certain distinguishable external markers such as coat color or eye color with homozygous and heterozygous states by placing a transgene responsible for such external marker phenotype in linkage with mutated fertility related gene. One such mouse strain ‘morc’ [Watson, M. L. et al., Identification of morc (microrchidia), a mutation that results in arrest of spermatogenesis at an early meiotic stage in the mouse”, Proc. Natl. Acad. Sci. 95: 14361-14366 (1998);] is available from The Jackson Laboratory, Bar Harbor, Me.

In another aspect of the invention, homozygous ES cells having permanent or conditional defects in the genes identified above are aggregated or injected along with genetically modified foreign cells into wild type diploid or tetraploid embryos or blastocysts. These ES cells can be made by standard protocols of gene targeting and are used to provide viable progenitors for non-germ line cells and increase the chances of successfully generating a viable chimeric blastocyst capable of growing into a whole animal.

In yet another aspect of the invention, we provide methods for generating genetically engineered animals, where the fertility related genes or their regulatory mechanisms can be regulated or conditionally mutated by using inducible promoters. This can be achieved by using standard inducible promoter systems including but not limited to tet-on and tet-off systems. As one example of how this can be achieved, one can introduce LoxP sites within or around the fertility related gene/s or in their regulatory mechanism/s such that the tetracycline mediated induction of Cre recombinase placed under tet-inducible promoter brings about mutation or dysfunction of the fertility related gene/s. The inducible system is necessary because it allows these genetically engineered mice to breed normally in spite of their being homozygous for mutation of fertility related gene/s and thereby allowing them to produce virtually all blastocysts/early stage embryos homozygous for the fertility related gene mutation or heterozygous for mutation in genes such as protamine-1 and protamine-2, where even heterozygous mutants are sterile. When the injection or aggregation experiment is planned, blastocysts or embryos derived from such animals can be incubated prior to or after the injection/aggregation experiment for a specific period of time in a medium containing tetracycline to render all blastocyst/embryo derived cells mutated for fertility related gene/s. Alternatively, the injection or aggregation experiment can be carried out using traditional method but following their birth, chimeric animals can be fed with tetracycline in their diet/water, so that all donor blastocyst/embryo derived cells in the chimeric animal are mutated for fertility related genes.

In yet another aspect, the invention provides a method for generating 100% homozygous blastocysts mutated for the fertility related gene(s) in contrast to 50% or less homozygous mutant blastocysts/embryos generated from mice with fertility related defects described earlier. This is achieved by first generating conditional knock-out mice for the fertility related gene(s). The conditional knock-out mouse is made by flanking a gene or a part of a gene, particularly the regulatory part, with recombinase recognition sites such as LoxP or Frt. Subsequently, a mutant male, which is homozygous for the conditional knock-out mutation but possesses at least one functionally active mutated allele of the fertility related gene is mated with a homozygous female which is also transgenic for the relevant recombinase gene. With such a mating, both alleles of the fertility related gene will be rendered functionally inactive in the resulting zygotes due to recombination so that all male blastocysts will be sterile. When ES cells are injected in such blastocysts, only ES cells will contribute to the germ line of the male chimera. The recombinase gene is preferably placed under a promoter which allows it to be expressed in the early embryonic stages. This method works equally well in female sterile mutant models with relevant changes.

In yet another aspect of invention, we provide a method for rendering wild type blastocysts or early stage embryos sterile for reproduction by incubating or injecting them with certain substances including but not limited to certain chemicals, antibodies and anti-sense RNA molecules which render the fertility related gene/s or gene products dysfunctional.

The improvement taught by the present invention can be applied to any species of animal which is amenable to, or in the future becomes amenable to, the conventional process of generating a genetically modified animal from blastocysts/embryos/ES cells injected or aggregated with foreign cells. This includes, but is not limited to, mouse, rat, hamster, cattle, goat, sheep and pig.

Foreign cells that may be used in the methods of the invention are cells which are capable of differentiation into germ line cells when introduced into a host blastocyst or aggregated with early stage embryos. This includes, but is not limited to, embryonic stem (ES) cells, Embryonic germ (EG) cells, teratocarcinoma stem cells and primordial germ cells (PGCs). These foreign cells carry the genetic modification(s) which are desired in the whole animal in either a heterozygous or homozygous state.

While the improvement taught herein can be used to create an animal having any desired genetic modification, it is particularly useful in the creation of knock-out, conditional knock-out and knock-in animals.

Various publications, patent applications and patents are cited herein, the disclosures of which are incorporated by reference in their entireties.

1. In a method for generating an animal having a desired genetic modification by injection or aggregation of foreign cells having said desired genetic modification into a blastocyst or embryo and subsequent growth and selection of animals from said blastocyst or embryo that have incorporated said foreign cells into their germ line, an improvement comprising the use of a source animal with a permanent or conditional defect in its germ line cells as the source of said blastocyst or embryo.

2. The method of claim 1 wherein said source animal has a permanent or conditional defect in a process selected from the group consisting of spermatogenesis, oogenesis, germ cell development, germ cell migration, and fertilization.

3. The method of claim 2 wherein said source animal has a defect in spermatogenesis.

4. The method of claim 3 wherein said source animal has a permanent or conditional defect in one or more genes selected from the group consisting of Calmegin, Fertilin beta, CatSper, Cyclin A1, Hsp 70-2, Scp3, A-myb, ACE, Ahch, Dazl, Trf2, Jsd, or in their regulatory mechanism/s.

5. The method in claim 3 wherein said source animal is a mouse selected from the group consisting of mutant strains Morc, W/Wv, Sl/Sld and XoSxrb.

6. The method of claim 2 wherein said process is selected from the group consisting of germ cell development and germ cell migration.

7. The method of claim 6 wherein said source animal has a permanent or conditional defect in one or more genes or their regulatory mechanisms selected from the group consisting of Smad1, Smad5, Bmp4/Bmp8b, E-cadherin, Fancc, Bcl-x/Bax and Beta1-integrin.

8. The method of claim 2 wherein said process is selected from the group consisting of oogenesis and fertilization.

9. The method of claim 8 wherein said source animal has a defect in the Cd9 gene or in its regulatory mechanisms.

10. The method of claim 1 wherein said source animal is selected from the group consisting of mouse, rat, hamster, cow, goat, sheep and pig.

11. The method of claim 1 wherein said foreign cells are selected from the group consisting of ES cells, EG cells, teratocarcinoma stem cells and primordial germ cells.

12. The method of claim 1 wherein said desired genetic modification comprises a defect in a gene or its regulatory mechanism which renders said gene completely, partially or conditionally nonfunctional.

13. A chimeric blastocyst or aggregate comprising cells from a source animal and foreign cells having a desired genetic modification, wherein said source animal has a permanent or conditional defect in its germ line cells.

14. In a method for generating a chimeric blastocyst or aggregate comprising cells from a source animal and foreign cells having a desired genetic modification useful in the generation of an animal having the desired genetic modification, an improvement comprising the use of a source animal with a permanent or conditional defect in its germ line cells.

15. A method for generating an animal having a desired genetic modification comprising the injection or aggregation of:
a. foreign cells having said desired genetic modification, and
b. ES cells that have a defect in certain genes involved in development or migration of germ line cells or in fertilization into wild type diploid or tetraploid blastocysts or embryos and subsequent growth and selection of animals from said blastocysts or embryos that have incorporated said foreign cells into their germ line.

16. A method for generating an inducibly sterile animal by placing a fertility related gene or the regulation of said fertility related gene under the direct or indirect control of an inducible system.

17. The method of claim 16 wherein said inducible system is the tet-on and tet-off system.

18. A method for rendering wild type blastocysts or early stage embryos sterile for reproduction by incubating or injecting them with certain substances including but not limited to certain chemicals, antibodies and anti-sense RNA molecules which render the fertility related gene/s or gene products dysfunctional.
